(19) United States
(12) Patent Application Publication
(10) Pub. No.: US 2011/0077539 A1
(43) Pub. Date: Mar. 31, 2011

(54) Methods and Compositions for Prediction of Risk for Sudden Death in Long QT Syndrome

(76) Inventors: Alfred L. George, Nashville, TN (US); Peter J. Schwartz, Milano (IT); Lia Crotti, Pavia (IT)

(21) Appl. No.: 12/892,410

(22) Filed: Sep. 28, 2010

Related U.S. Application Data
(60) Provisional application No. 61/247,085, filed on Sep. 30, 2009.

(51) Int. Cl. A61B 5/0402; C12Q 1/68

(52) U.S. Cl. 600/509; 435/6

(57) ABSTRACT

The invention generally concerns methods and compositions for screening individuals for predicting increased sudden death risk in a population of subjects having long QT syndrome (LQTS) or subjects at risk for sudden infant death syndrome (SIDS) by examining single nucleotide polymorphisms (SNPs) in the NOS1AP gene. In particular, the minor alleles for both rs16847548 and rs4657139 predicted an increased risk for sudden death in LQTS, while subjects carrying the GG or TG genotype (G is the minor allele) for rs10494366 were at increased risk of sudden death from SIDS. This information permits more attentive monitoring and/or prophylactic treatments of high risk individuals.

This application claims benefit of priority to U.S. Provisional Application Ser. No. 61/247,085, filed Sep. 30, 2009, the entire contents of which are hereby incorporated by reference.

This invention was made with government support under grant no. R01-HL068880 awarded by National Institutes of Health/National Heart Lung and Blood Institute. The government has certain rights in the invention

### BACKGROUND OF THE INVENTION

1. Field of the Invention

The present invention relates generally to the fields of genetics and medicine. Specifically, the invention relates to compositions and methods for predicting the risk of sudden death in a population of subjects having long QT syndrome (LQTS) or subjects at risk for sudden infant death syndrome (SIDS) by examining single nucleotide polymorphisms (SNPs) in the NOS1AP gene.

2. Description of Related Art

The congenital long-QT syndrome (LQTS) is an inherited disorder of abnormal myocardial repolarization in which there is a high risk for potentially lethal cardiac arrhythmias (Schwartz et al., 2009). The disorder is caused by mutations in several genes most of which encode ion channel subunits involved in the regulation of the cardiac action potential. The most common form of LQTS (LQT1) is caused by mutations in KCNQ1, a gene encoding the pore-forming subunit of potassium channels responsible for the slow cardiac delayed rectifier current (Wang et al., 1996). In many families, LQTS exhibits incomplete penetrance and variable expressivity, which suggest the existence of factors other than the primary mutation that can modify the probability of symptoms (Priori et al., 1999; Schwartz et al., 2003; Crotti et al., 2005; Westenskow et al., 2004). Identification of genetic modifiers of LQTS would lead to improved risk stratification among mutation carriers and could also provide information about the risk for life-threatening arrhythmias in more common conditions, such as acute myocardial infarction and congestive heart failure.

A prolonged QT interval is a surrogate measurement of prolonged ventricular repolarization and is a widely recognized subclinical marker for increased risk of life-threatening cardiac arrhythmia in congenital and acquired forms of LQTS and after a myocardial infarction (Schwartz and Wolf, 1978; Chugh et al., 2009). A recent genome wide association study identified genetic variation in NOS1AP, which encodes a nitric oxide synthase adaptor protein, as a contributor to QT interval duration in the general population (Arking et al., 2006). Although the absolute quantitative effect of NOS1AP variants on the QT interval in healthy subjects was small, explaining up to 1.5% of QT interval variation, the replication of this finding in several distinct populations demonstrated that the association is robust (Aarnoudse et al., 2007; Post et al., 2007; raitakari et al., 2008; Tobin et al., 2008; Lehtinen et al., 2008; Arking et al., 2009; Eijgelsheim et al., 2009; Newton-Cheh et al., 2009; Pfeufer et al., 2009). Further analyses have found an association between NOS1AP and risk for sudden death in a general population (Kao et al., 2009) and increased cardiovascular mortality in users of calcium channel blockers (Becker et al., 2009). Whether genetic variation in NOS1AP contributes to the risk of sudden death in congenital LQTS is not known.

### SUMMARY OF THE INVENTION

Thus, in accordance with the present invention, there is provided a method of predicting increased risk of sudden death from long QT syndrome (LQTS) or sudden infant death syndrome (SIDS) comprising (a) obtaining a DNA-containing sample from a subject with congenital LQTS; (b) assessing the structure of the NOS1AP gene at rs16847548 and/or rs4657139; and (c) making a prediction of risk based on the structure of the NOS1AP gene at rs16847548 and/or rs4657139; wherein the presence of a rs16847548 C allele and/or a rs4657139 A allele indicates that the subject is at increased risk of experiencing sudden death from LQTS or SIDS as compared to a subject having a rs16847548 T allele and/or a rs4657139 T allele. One may also assess a structure that is determined to be in linkage disequilibrium with rs16847548 and/or rs4657139.

The method may further comprising examining at least one additional risk factor for LQTS and/or SIDS for the subject, such as presence of a mutation in an LQTS risk gene, or a LQTS risk score of 3 or higher, or a relative being diagnosed with LQTS.

Assessing the structure may comprise sequencing, primer extension, differential, and/or a 5′-nucleotidase assay. The method may further comprise amplifying at least a portion of the NOS1AP gene, such as polymerase chain reaction.

The may further comprise making a decision regarding monitoring or treatment of the subject, such as implantation of an automated internal defibrillator or internal recording device (‘event recorder’).

The subject may be a newborn of less than about one month of age, an infant of about one month to about 3 years of age, or an adult. The subject may have a rs16847548 C allele and a rs4657139 A allele, a rs16847548 T allele and a rs4657139 T allele, a rs16847548 C allele and a rs4657139 T allele, or a rs16847548 T allele and a rs4657139 A allele.

The method may further comprising treating the subject when determined to be at increased risk of sudden death with an anti-arrhythmic compound, such as with a β blocker, a sodium channel blocker, or potassium channel modulator.

The method may further comprising diagnosing the the subject as having LQTS, such as by genetic testing for an LQTS mutation in DNA from the subject, by taking a family history from the subject, or by performing a physical examination of the subject, including an electrocardiogram.

It is contemplated that any method or composition described herein can be implemented with respect to any other method or composition described herein. Similarly, any embodiment discussed with respect to one aspect of the invention may be used in the context of any other aspect of the invention.

Throughout this application, the term “about” is used to indicate that a value includes the standard deviation of error for the device or method being employed to determine the value.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawing forms part of the present specification and is included to further demonstrate certain aspects of the present invention. The invention may be better understood by reference to the drawing in combination with the detailed description of specific embodiments presented herein.

FIGS. 1A-B—Variants and linkage disequilibrium (LD) in NOS1AP. (FIG. 1A) Minor allele frequencies for each NOS1AP variant observed in the study population and in the western European ancestry sample of the HapMap Project (numbers in parentheses). The minor allele listed on top. (FIG. 1B) Pairwise LD between 5 NOS1AP variants determined using HapMap data for white Europeans. The value within each diamond represents the pairwise correlation between variants (measured as r²) defined by the top left and the top right sides of the diamond. The approximate location of NOS1AP exons 1 and 2 are shown as black squares.

### DETAILED DESCRIPTION OF ILLUSTRATIVE EMBODIMENTS

Inherited arrhythmia susceptibility, such as in LQTS, is a known cause of sudden cardiac death especially in young adults and children. Accurate risk stratification is critically important for effective utilization of preventive strategies, but even among subjects found to carry the same LQTS mutation the probability of life-threatening cardiac events can vary considerably. This clinical heterogeneity can be explained in rare cases by compound heterozygosity (Schwartz et al., 2003; Westenskow et al., 2004), but common genetic factors other than the primary disease-causing mutation are also likely modifiers of arrhythmic risk. Defining genetic modifiers of LQTS could have a significant impact on the accuracy of individual risk stratification.

The inventors tested the hypothesis that NOS1AP is a genetic modifier of LQTS in a South African population segregating the KCNQ1-A341V mutation and exhibiting variable disease expression among mutation carriers (Brink et al., 2005). This population is particularly well-suited for testing genetic modifier hypotheses because all at-risk subjects share the same disease-causing mutation, a feature that offers advantages over using LQTS populations having heterogeneous mutations in multiple different genes, a factor known to confer varying levels of arrhythmia risk (Priori et al., 2003; Schwartz et al., 2001; Moss et al., 2007).

The main finding of the study is that common NOS1AP variants are modifiers of the clinical severity of congenital LQTS and are associated with a greater chance of having a more prolonged QT interval in mutation carriers. This is the first evidence, demonstrated in subjects sharing the same mutation, that NOS1AP variants are associated with a greater risk for cardiac arrest and sudden death in LQTS. These findings may contribute to the refinement of individual risk stratification in LQTS and help prompt consideration of new mechanistic hypotheses of arrhythmia susceptibility in this disease.

The inventors tested NOS1AP as a candidate LQTS modifier gene in a large group of subjects carrying the same mutation as the underlying cause for arrhythmia susceptibility. This unique study design eliminated the confounding effects of genetic and allelic heterogeneity that is present when a study involves multiple different disease-causing mutations that are known to carry widely different arrhythmic risk (Priori et al., 2003; Moss et al., 2007). The inventors specifically studied an LQT1 founder population harboring a mutation in KCNQ1 (A341V) that exhibits a wide range of QTc values and clinical manifestations (Brink et al., 2005; Crotti et al., 2007). The novel finding is that the minor allele at common NOS1AP variant rs16847548 is associated with the risk of cardiac events, and—importantly—with the occurrence of life-threatening events. These findings are in agreement with the association of rs16847548 with the risk of sudden cardiac death demonstrated in a general population of white Americans (Kao et al., 2009).

The inventors also observed an association between the minor allele of two NOS1AP variants (rs4657139 and rs16847548) with the probability of having QTc duration in the top 40% of all QTc values among mutation carriers. Although this observation may not seem surprising at first glance given the prior associations with QT duration in general populations, they regarded this finding as unexpected for the following reason. Whereas a modest effect on QT duration was detectable in very large populations having mean QT values within a normal range, it was unclear whether an association of NOS1AP with QT could be detected in an LQTS population with a mean QTc value close to 500 ms because of a predicted “ceiling effect” in which the contribution of the underlying mutation to QT interval duration might dwarf any minor effect of NOS1AP variation. This is why they were impressed by the fact that, even with a small sample size and analyzing QTc as a categorical variable, the association of rs4657139 and rs16847548 with QTc could be demonstrated in this LQTS population.

There is scant information regarding the biological influence of NOS1AP genetic variation on function or expression of the gene and how this relates to effects on the QT interval or risk for cardiac events. Because NOS1AP variants associated with the QT interval are located in non-coding regions of the gene, the presumption is that transcriptional influences exerted by cis-acting elements may differ among alleles. Work from laboratories investigating genetic associations between NOS1AP and schizophrenia have elucidated potential transcriptional effects of certain common variants by using in vitro reporter-gene experiments. Specifically, the A allele of one variant (rs12742393) located in the second intron enhances binding of a presumed nuclear transcription factor and drives greater transcriptional activity of the NOS1AP promoter in human neural cell lines (Wratten et al., 2009). Similar studies using cardiac tissue have not been published.

Although the potential transcriptional effects of NOS1AP variants on gene expression in heart are not known, Chang et al. (2008) found that over-expression of the NOS1AP gene product (CAPON) in isolated guinea pig myocytes causes attenuation of L-type calcium current, a slight increase in rapid delayed rectifier current (I_{Kr}) and shortening of action potentials. These observations suggest plausible cellular mechanisms that might explain the inventors' findings in this study. For example, if one postulates that genetic variants in NOS1AP impair expression and lead to lower levels of CAPON, then based on the study by Chang et al. (2008), one might expect increased L-type calcium current with associated arrhythmogenic consequences. Further, as calcium current is enhanced by sympathetic activation, a greater effect would be anticipated in conditions associated with augmented catecholamine release such as physical or emotional stress, the predominant clinical circumstances associated with lethal arrhythmic episodes in LQT1 (Schwartz et al., 2001).

By studying this highly unique founder population, one can take advantage of genetic homogeneity, essential for assessing the contribution of potential modifiers. However, the limitation of this approach is that the feasibility of performing a comparable replication study is extremely low. Whether these findings made in this founder population will apply to LQTS mutation carriers in other populations remains to be determined. Further, because of the restricted size of the study population, the statistical power of the data was insufficient to test all known NOS1AP variants previously associated with variation of QT duration or an unlimited number of other candidate variants. A much larger population would have been required to examine effects of NOS1AP variants on the QT interval analyzed as a continuous variable. Ascertainment bias could have influenced the results, because subjects carrying both KCNQ1-A341V and the NOS1AP risk allele have a greater probability of sudden death. But, this potential bias would have actually diminished chances of observing a significant association. This suggests conceptually that the findings reported here are robust to any selection bias imposed by the greater risk of death in such carriers.

In addition, the inventors have demonstrated that long QT syndrome (LQTS) contributes to Sudden Infant Death Syndrome (SIDS), the leading cause of mortality in the first year of life. A prolonged QT interval in the first week is associated with a significantly higher risk of SIDS and 10% of SIDS victims carry functionally significant genetic variants in LQTS genes. These results show that the minor allele of NOS1AP rs10494366, previously correlated with QT interval duration, is associated with an increased risk of SIDS in a Norwegian cohort.

These and other aspects of the invention are discussed in greater detail in the following disclosure.

### I. LONG QT SYNDROME

The long QT syndrome (LQTS) is a rare, congenital heart condition with delayed repolarization following depolarization (excitation) of the heart, associated with syncope (fainting) due to ventricular arrhythmias, possibly of type torsade de pointes, which can deteriorate into ventricular fibrillation and ultimately sudden death. Arrhythmia in individuals with LQTS is often associated with exercise or excitement.

The first documented case of LQTS was described in Leipzig by Meissner in 1856, where a deaf mute girl died after her teacher yelled at her. When the parents were told about her death, they told that her older brother who also was deaf mute died after a terrible fright. This was before the ECG was invented, but is likely the first described case of Jervell and Lange-Nielsen syndrome. In 1957, the first case documented by ECG was described by Anton Jervell and Fred Lange-Nielsen. Romano, in 1963, and Ward, in 1964, separately described the more common variant of Long QT syndrome with normal hearing, later called Romano-Ward syndrome. The establishment of the International Long-QT Syndrome Registry in 1979 allowed numerous pedigrees to be evaluated in a comprehensive manner. This helped in detecting many of the numerous genes involved.

A number of syndromes are associated with LQTS. The Jervell and Lange-Nielsen syndrome (JLNS) is an autosomal recessive form of LQTS with associated congenital deafness. It is caused specifically by mutation of the KCNE1 and KCNQ1 genes. In untreated individuals with JLNS, about 50 percent die by the age of 15 years due to ventricular arrhythmias. Romano-Ward syndrome is an autosomal dominant form of LQTS that is not associated with deafness. The diagnosis is clinical and is now less commonly used in centres where genetic testing is available, in favour of the LQT1 to 10 scheme given above.

Individuals with LQTS have a prolongation of the QT interval on the ECG. The QRS complex corresponds to ventricular depolarization while the T wave corresponds to ventricular repolarization. The QT interval is measured from the Q point to the end of the T wave. While many individuals with LQTS have persistent prolongation of the QT interval, some individuals do not always show the QT prolongation; in these individuals, the QT interval may prolong with the administration of certain medications.

A. Acquired LQTS

More common than the various congenital causes of long QT syndrome are acquired causes. They can be divided into two main categories—those due to disturbances in blood electrolytes (hypokalemia, hypomagnesemia, hypocalcemia) and those due to various drugs, including Anti-arrhythmic drugs (Quinidine, Amiodarone, Sotalol, Procainamide, Ranolazine), Anti-histamines (terfenadine, astemizole), Macrolide antibiotics (Erythromycin), certain Fluoroquinolone antibiotics, Major tranquilizers, Tricyclic antidepressants, Gastrointestinal Motility agents (Cisapride, Domperidone), Antipsychotic drugs (Haloperidol, Quetiapine, Thioridazine, Droperidol) and Analgesics (Methadone, LAAM).

Just as with the congenital causes of the LQTS, the acquired causes may also lead to the potentially lethal arrythmia known as Torsade de Pointes. Treatment is straightforward—replace any deficient electrolytes if present and stop any culprit drugs if the patient is using one (or more).

Given its relatively high frequency of use, its tendency for drug-drug interaction, and its inherent ability to prolong the QT interval, the macrolide antibiotic erythromycin is probably the most prevalent cause of acquired long QT syndrome. Indeed, use of erythromycin is associated with a rate of death more than double that of use of other antibiotics.

In addition to the two major categories listed above, it should be noted that there are also some miscellaneous causes of QT prolongation such as anorexia nervosa, hypothyroidism, HIV infection, and myocardial infarction.

B. Congenital LQTS

Genetic LQTS can arise from mutation to one of several genes. These mutations tend to prolong the duration of the ventricular action potential (APD), thus lengthening the QT interval. LQTS can be inherited in an autosomal dominant or an autosomal recessive fashion. The autosomal recessive forms of LQTS tend to have a more severe phenotype, with some variants having associated syndactyly (LQT8) or congenital neural deafness (LQT1). A number of specific genes loci have been identified that are associated with LQTS. Genetic testing for LQTS is clinically available and may help to direct appropriate therapies (Overview of LQTS Genetic Testing). The most common causes of LQTS are mutations in the genes KCNQ1 (LQT1), KCNH2 (LQT2), and SCN5A (LQT3); the following is a list of all known genes associated with LQTS.

TABLE 1
LTQS GENE SUMMARY

| Type | OMIM | Mutation | Notes |
|---|---|---|---|
| LQT1 | 192500 | α subunit of the | The current through the heteromeric channel |
| | | slow delayed | (KvLQT1 + minK) is known as I_{Ks}. These |
| | | rectifier potassium | mutations often cause LQT by reducing the |
| | | channel (KvLQT1 | amount of repolarizing current. This repolarizing |
| | | or KCNQ1) | current is required to terminate the action potential, |
| | | | leading to an increase in the action potential |
| | | | duration (APD). These mutations tend to be the |
| | | | most common yet least severe. |
| LQT2 | 152427 | α subunit of the | Current through this channel is known as I_{Kr}. This |
| | | rapid delayed | phenotype is also probably caused by a reduction |
| | | rectifier potassium | in repolarizing current. |
| | | channel (HERG + | |
| | | MiRP1) | |
| LQT3 | 603830 | α subunit of the | Current through this channel is commonly referred |
| | | sodium channel | to as I_{Na}. Depolarizing current through the channel |
| | | (SCN5A) | late in the action potential is thought to prolong |
| | | | APD. The late current is due to the failure of the |
| | | | channel to remain inactivated. Consequently, it can |
| | | | enter a bursting mode, during which significant |
| | | | current enters abruptly when it should not. These |
| | | | mutations are more lethal but less common. |
| LQT4 | 600919 | anchor protein | LQT4 is very rare. Ankyrin B anchors the ion |
| | | Ankyrin B | channels in the cell. |
| LQT5 | 176261 | β subunit MinK (or | — |
| | | KCNE1) which | |
| | | coassembles with | |
| | | KvLQT1 | |
| LQT6 | 603796 | β subunit MiRP1 | — |
| | | (or KCNE2) which | |
| | | coassembles with | |
| | | HERG | |
| LQT7 | 170390 | potassium channel | The current through this channel and KCNJ12 |
| | | KCNJ2 (or Kᵢᵣ2.1) | (Kᵢᵣ2.2) is called I_{Kl}. LQT7 leads to Andersen- |
| | | | Tawil syndrome. |
| LQT8 | 601005 | α subunit of the | Leads to Timothyis syndrome. |
| | | calcium channel | |
| | | Cav1.2 encoded by | |
| | | the gene | |
| | | CACNA1c. | |
| LQT9 | 611818 | Caveolin 3 | — |
| LQT10 | 611819 | SCN4B | — |
| LQT11 | 611820 | AKAP9 | — |
| LQT12 | 601017 | SNTA1 | — |

Drug induced LQT is usually a result of treatment by anti-arrhythmic drugs such as amiodarone or a number of other drugs that have been reported to cause this problem (e.g., cisapride). Some anti-psychotic drugs, such as Haloperidol and Ziprasidone, have a prolonged QT interval as a rare side effect. Genetic mutations may make one more susceptible to drug-induced LQT.

LQT1. LQT1 is the most common type of long QT syndrome, making up about 30 to 35 percent of all cases. The LQT1 gene is KCNQ1 which has been isolated to chromosome 11p15.5. KCNQ1 codes for the voltage-gated potassium channel KvLQT1 that is highly expressed in the heart. It is believed that the product of the KCNQ1 gene produces an alpha subunit that interacts with other proteins (particularly the minK beta subunit) to create the I_{Ks} ion channel, which is responsible for the delayed potassium rectifier current of the cardiac action potential.

Mutations to the KCNQ1 gene can be inherited in an autosomal dominant or an autosomal recessive pattern in the same family. In the autosomal recessive mutation of this gene, homozygous mutations in KVLQT1 leads to severe prolongation of the QT interval (due to near-complete loss of the I_{Ks} ion channel), and is associated with increased risk of ventricular arrhythmias and congenital deafness. This variant of LQT1 is known as the Jervell and Lange-Nielsen syndrome. Most individuals with LQT1 show paradoxical prolongation of the QT interval with infusion of epinephrine. This can also unmark latent carriers of the LQT1 gene. Many missense mutations of the LQT1 gene have been identified. These are often associated with a high frequency of syncopes but less sudden death than LQT2.

LQT2. The LQT2 type is the second most common gene location that is affected in long QT syndrome, making up about 25 to 30 percent of all cases. This form of long QT syndrome most likely involves mutations of the human ether-a-go-go related gene (HERG) on chromosome 7. The HERG gene (also known as KCNH2) is part of the rapid component of the potassium rectifying current (I_{Kr}). (The I_{Kr} current is mainly responsible for the termination of the cardiac action potential, and therefore the length of the QT interval.) The normally functioning HERG gene allows protection against early after depolarizations (EADs).

Most drugs that cause long QT syndrome do so by blocking the I_{Kr} current via the HERG gene. These include erythromycin, terfenadine, and ketoconazole. The HERG channel is very sensitive to unintended drug binding due to two aromatic amino acids, the tyrosine at position 652 and the phenylalanine at position 656. These amino acid residues are poised so a drug binding to them will block the channel from conducting current. Other potassium channels do not have these residues in these positions and are therefore not as prone to blockage.

LQT3. The LQT3 type of long QT syndrome involves mutation of the gene that encodes the alpha subunit of the Na⁺ ion channel. This gene is located on chromosome 3p21-24, and is known as SCN5A (also hH1 and Naᵥ1.5). The mutations involved in LQT3 slow the inactivation of the Na⁺ channel, resulting in prolongation of the Na⁺ influx during depolarization. Paradoxically, the mutant sodium channels inactivate more quickly, and may open repetitively during the action potential.

A large number of mutations have been characterized as leading to or predisposing to LQT3. Calcium has been suggested as a regulator of SCN5A, and the effects of calcium on SCN5A may begin to explain the mechanism by which some these mutations cause LQT3. Furthermore, mutations in SCN5A can cause Brugada syndrome, cardiac conduction disease and dilated cardiomyopathy. Rarely some affected individuals can have combinations of these diseases.

LQT5. LTQ5 is an autosomal dominant relatively uncommon form of LQTS. It involves mutations in the gene KCNE1 which encodes for the potassium channel beta subunit MinK. In its rare homozygous forms it can lead to Jervell and Lange-Nielsen syndrome

LQT6. LTQ6 is an autosomal dominant relatively uncommon form of LQTS. It involves mutations in the gene KCNE2 which encodes for the potassium channel beta subunit MiRP1, constituting part of the I_{Kr} repolarizing K⁺ current.

LQT7. Andersen-Tawil syndrome is an autosomal dominant form of LQTS associated with skeletal deformities. It involves mutation in the gene KCNJ2 which encodes for the potassium channel protein Kir 2.1. The syndrome is characterized by Long QT syndrome with ventricular arrhythmias, periodic paralysis and skeletal developmental abnormalities as clinodactyly, low-set ears and micrognathia. The manifestations are highly variable.

LQT8. Timothy's syndrome is due to mutations in the calcium channel Cav1.2 encoded by the gene CACNA1c. Since the Calcium channel Cav1.2 is abundant in many tissues, patients with Timothy's syndrome have many clinical manifestations including congenital heart disease, autism, syndactyly and immune deficiency.

LQT9. This newly discovered variant is caused by mutations in the membrane structural protein, caveolin-3. Caveolins form specific membrane domains called caveolae in which among others the Naᵥ1.5 voltage-gated sodium channel sits. Similar to LQT3, these particular mutations increase so-called ‘late’ sodium current which impairs cellular repolarization.

LQT10. This novel susceptibility gene for LQT is SCN4B encoding the protein Naᵥβ4, an auxiliary subunit to the pore-forming Naᵥ1.5 (gene: SCN5A) subunit of the voltage-gated sodium channel of the heart. The mutation leads to a positive shift in inactivation of the sodium current, thus increasing sodium current. Only one mutation in one patient has so far been found.

All forms of the long QT syndrome involve an abnormal repolarization of the heart. The abnormal repolarization causes differences in the “refractoriness” of the myocytes. After-depolarizations (which occur more commonly in LQTS) can be propagated to neighboring cells due to the differences in the refractory periods, leading to re-entrant ventricular arrhythmias. It is believed that the so-called early after-depolarizations (EADs) that are seen in LQTS are due to re-opening of L-type calcium channels during the plateau phase of the cardiac action potential. Since adrenergic stimulation can increase the activity of these channels, this is an explanation for why the risk of sudden death in individuals with LQTS is increased during increased adrenergic states (i.e., exercise, excitement), especially since repolarization is impaired. Normally during adrenergic states, repolarizing currents will also be enhanced to shorten the action potential. In the absence of this shortening and the presence of increased L-type calcium current, EADs may arise.

The so-called delayed after-depolarizations (DADs) are thought to be due to an increased Ca²⁺ filling of the sarcoplasmic reticulum. This overload may cause spontaneous Ca²⁺ release during repolarization, causing the released Ca²⁺ to exit the cell through the 3Na⁺/Ca²⁺-exchanger which results in a net depolarizing current.

The diagnosis of LQTS is not easy since 2.5% of the healthy population have prolonged QT interval, and 10-15% of LQTS patients have a normal QT interval. A commonly used criterion to diagnose LQTS is the LQTS “diagnostic score.” The score is calculated by assigning different points to various criteria (listed below). With 4 or more points the probability is high for LQTS, and with 1 point or less the probability is low. Two or 3 points indicates intermediate probability:
QTc (Defined as QT interval/square root of RR interval)
   >=480 msec—3 points
   460-470 msec—2 points
   450 msec and male gender—1 point
Torsades de Pointes ventricular tachycardia—2 points
T wave alternans—1 point
Notched T wave in at least 3 leads—1 point
Low heart rate for age (children)—0.5 points
Syncope (one cannot receive points both for syncope and Torsades de pointes) with stress—2 points
   without stress—1 point
   Congenital deafness—0.5 points
Family history (the same family member cannot be counted for LQTS and sudden death)
   Other family members with definite LQTS—1 point
   Sudden death in immediate family (members before the age 30)—0.5 points

### II. SUDDEN INFANT DEATH SYNDROME

Sudden infant death syndrome (SIDS) or crib death is a syndrome marked by the sudden death of an infant that is unexpected by history and remains unexplained after a thorough forensic autopsy and a detailed death scene investigation. The term cot death is often used in the United Kingdom, Ireland, Australia, India, South Africa and New Zealand. Typically the infant is found dead after having been put to bed, and exhibits no signs of having suffered.

SIDS is a diagnosis of exclusion. It should only be applied to an infant whose death is sudden and unexpected, and remains unexplained after the performance of an adequate postmortem investigation including an autopsy, investigation of the scene and circumstances of the death, and exploration of the medical history of the infant and family.

SIDS was responsible for 0.543 deaths per 1,000 live births in the U.S. in 2005. It is responsible for far fewer deaths than congenital disorders and disorders related to short gestation, though it is the leading cause of death in healthy infants after one month of age. SIDS deaths in the U.S. decreased from 4,895 in 1992 to 2,247 in 2004. But, during a similar time period, 1989 to 2004, SIDS being listed as the cause of death for sudden infant death (SID) decreased from 80% to 55%.

Epidemiology of SIDS and physiological evidence shows that infants who sleep on their back have lower arousal thresholds and less Slow-Wave Sleep (SWS) compared to infants who sleep on their stomachs. In human infants, sleep develops rapidly during early development. This development includes an increase in non-rapid eye movement sleep (NREM sleep) which is also called Quiet Sleep (QS) during the first 12 months of life in association with a decrease in rapid eye movement sleep (REM sleep) which is also known as Active Sleep (AS). In addition, slow wave sleep (SWS) which consists of Stage 3 and Stage 4 NREM sleep appears at 2 months of age, and it is theorized that some infants have a brain-stem defect which increases their risk of being unable to arouse from SWS (also called Deep Sleep) and therefore have an increased risk of SIDS due to their increased inability to arouse from SWS.

Studies have shown that preterm infants, full-term infants, and older infants have greater time periods of quiet sleep and also decreased time awake when they are positioned to sleep on their stomachs. In both human infants and rats, arousal thresholds have been shown to be at higher levels in the Electroencephalography (EEG) during Slow-wave sleep

In 1992, a SIDS risk reduction strategy based upon lowering arousal thresholds during SWS was implemented by the American Academy of Pediatrics (AAP) which began recommending that healthy infants be positioned to sleep on their back (supine position) or side (lateral position), instead of their stomach (prone position), when being placed down for sleep. In 1994, a number of organizations in the United States combined to further communicate these non-prone sleep position recommendations. In 1996, the AAP further refined its sleep position recommendation by stating that infants should only be placed to sleep in the supine position and not in the prone or lateral positions.

Some conditions that may be undiagnosed and thus could be alternative diagnoses to SIDS include:

medium-chain acyl-coenzyme A dehydrogenase deficiency (MCAD deficiency)

infant botulism

long QT syndrome

infections with the bacterium Helicobacter pylori

shaken baby syndrome and other forms of child abuse

For example an infant with MCAD deficiency could have died by ‘classical SIDS” if found swaddled and prone with head covered in an overheated room where parents were smoking. Genes of susceptibility to MCAD and Long QT syndrome do not protect an infant from dying of classical SIDS. Therefore, presence of a susceptibility gene, such as for MCAD, means the infant may have died either from SIDS or from MCAD deficiency. It is impossible for the pathologist to distinguish between them.

Very little is certain about the possible causes of SIDS, and there is no proven method for prevention. Although studies have identified risk factors for SIDS, such as putting infants to bed on their stomachs, there has been little understanding of the syndrome's biological cause or causes. The frequency of SIDS appears to be a strong function of the infant's sex, age and ethnicity, and the education and socio-economic-status of the infant's parents.

According to a study published in 2007, babies who die of SIDS have abnormalities in the brain stem (the medulla oblongata), which helps control functions like breathing, blood pressure and arousal, and abnormalities in serotonin signaling. According to the National Institutes of Health, which funded the study, this finding is the strongest evidence to date that structural differences in a specific part of the brain may contribute to the risk of SIDS.

In a British study from 2008, researchers discovered that the common bacterial infections Staphylococcus aureus (staph) and Escherichia coli (E. coli) appear to be the cause of some cases of Sudden Infant Death Syndrome. Both bacteria were present at greater than usual concentrations in infants who died from SIDS. SIDS cases peak between eight and ten weeks after birth, which is also the time frame in which the antibodies that were passed along from mother to child are starting to disappear and babies have not yet made their own antibodies.

Listed below are several factors associated with increased probability of the syndrome:

Prenatal
maternal nicotine use (tobacco or nicotine patch)
inadequate prenatal care
inadequate prenatal nutrition
use of heroin
subsequent births less than one year apart
alcohol use
infant being overweight
mother being overweight
teen pregnancy
infant's sex (60% of SIDS cases occur in males)

Postnatal
mold exposure
low birth weight
exposure to tobacco smoke
prone sleep position (lying on the stomach)
not breastfeeding
elevated room temperature
excess bedding, clothing, soft sleep surface and stuffed animals
infant's age (incidence rises from zero at birth, is highest from two to four months, and declines towards zero at one year)
premature birth (increases risk of SIDS death by about 4 times)
anemia

### III. NOS1AP

Nitric oxide synthase 1 (neuronal) adaptor protein, also known as NOS1AP, is a human gene. This gene encodes a cytosolic protein that binds to the signaling molecule, neuronal nitric oxide synthase (nNOS). This protein has a C-terminal PDZ-binding domain that mediates interactions with nNOS and an N-terminal phosphotyrosine binding (PTB) domain that binds to the small monomeric G protein, Dexras1. Studies of the related mouse and rat proteins have shown that this protein functions as an adapter protein linking nNOS to specific targets, such as Dexras1 and the synapsins.

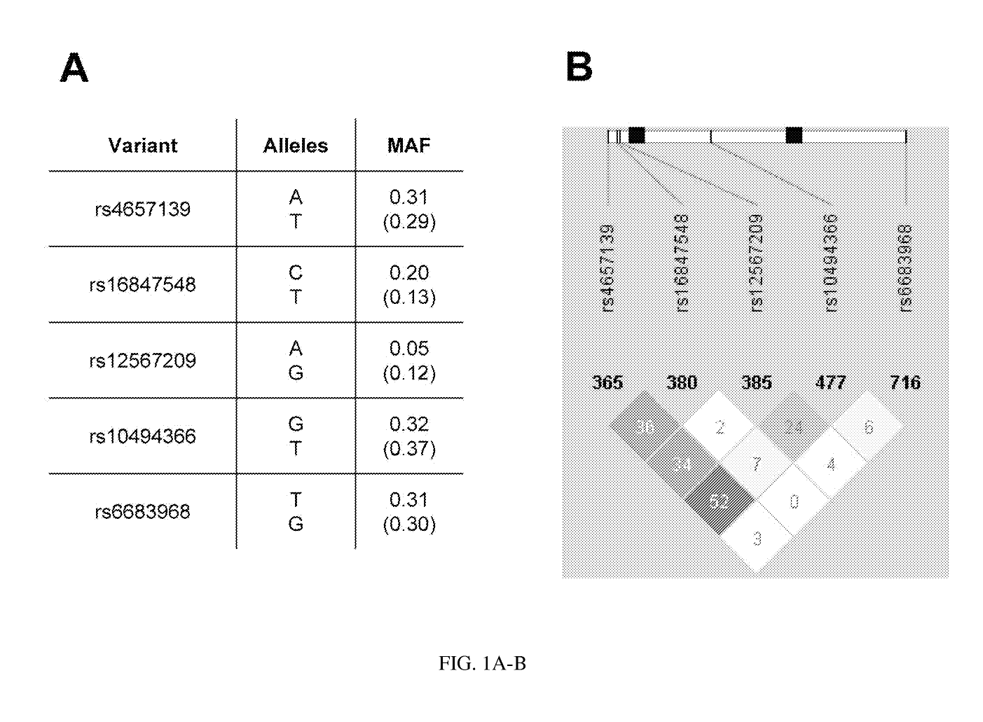

The accession no. for the human mRNA is NM 014697, and for the protein is NP 055512. The NOS1AP gene spans approximately 300 kilobases on human chromosome 1q23.3 and contains 10 exons. The locations of the various SNPs examined by the inventors in this study are shown in FIG. 1B. The SNPs predictive of sudden death risk are located within intron or putative regulatory regions of the gene.

### IV. NUCLEIC ACID DETECTION

Some embodiments of the invention concern identifying polymorphisms in sequences such a genomic DNA and mRNA, correlating to increased or decreased risk for sudden death. Thus, the present invention involves assays for identifying polymorphisms and other nucleic acid detection methods. It is contemplated that probes and primers can be prepared based on previously published sequences for each of he targets. Nucleic acids, therefore, have utility as probes or primers for embodiments involving nucleic acid hybridization. They may be used in diagnostic or screening methods of the present invention. General methods of nucleic acid detection methods are provided below, followed by specific examples employed for the identification of polymorphisms, including single nucleotide polymorphisms (SNPs).

A. Hybridization

The use of a probe or primer of between 7, 8, 9, 10, 11, 12, 13, 14, 15, 16, 17, 18, 19, 20, 21, 22, 23, 24, 25, 50, 60, 70, 80, 90, or 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC/0.1% SDS at 68° C. (Ausubel et al., 1989).

Conditions may be rendered less stringent by increasing salt concentration and/or decreasing temperature. For example, a medium stringency condition could be provided by about 0.1 to 0.25M NaCl at temperatures of about 37° C. to about 55° C., while a low stringency condition could be provided by about 0.15M to about 0.9M salt, at temperatures ranging from about 20° C. to about 55° C. Under low stringent conditions, such as moderately stringent conditions the washing may be carried out for example in 0.2×SSC/0.1% SDS at 42° C. (Ausubel et al., 1989). Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples. In other aspects, a particular nuclease cleavage site may be present and detection of a particular nucleotide sequence can be determined by the presence or absence of nucleic acid cleavage.

In general, it is envisioned that the probes or primers described herein will be useful as reagents in solution hybridization, as in PCR, for detection of expression or genotype of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

B. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples with or without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to the sequence flanking the target site of interest, or variants thereof, and fragments thereof are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,”” are conducted until a sufficient amount of amplification product is produced.

The amplification product may be detected, analyzed or quantified. In certain applications, the detection may be performed by visual means. In certain applications, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Application 2 202 328, and in PCT Application PCT/US89/01025, each of which is incorporated herein by reference in its entirety. Qbeta Replicase, described in PCT Application PCT/US87/00880, may also be used as an amplification method in the present invention.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application 329 822 disclose a nucleic acid amplification process involving cyclically synthesizing single-stranded RNA (ssRNA), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (ssDNA) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

C. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by spin columns and/or chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

D. Other Assays

Other methods for genetic screening may be used within the scope of the present invention, for example, to detect mutations in genomic DNA, cDNA and/or RNA samples. Methods used to detect point mutations include denaturing gradient gel electrophoresis (DGGE), restriction fragment length polymorphism analysis (RFLP), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

E. Specific Examples of Polymorphism Screening Methods

Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLPs have been widely used in human and animal genetic analyses.

Another class of polymorphisms is generated by the replacement of a single nucleotide. Such single nucleotide polymorphisms (SNPs) rarely result in changes in a restriction endonuclease site. Thus, SNPs are rarely detectable restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset alzheimer disease etc.

SNPs can be the result of deletions, point mutations and insertions and in general any single base alteration, whatever the cause, can result in a SNP. The greater frequency of SNPs means that they can be more readily identified than the other classes of polymorphisms. The greater uniformity of their distribution permits the identification of SNPs “nearer” to a particular trait of interest. The combined effect of these two attributes makes SNPs extremely valuable. For example, if a particular trait (e.g., inability to efficiently metabolize irinotecan) reflects a mutation at a particular locus, then any polymorphism that is linked to the particular locus can be used to predict the probability that an individual will be exhibit that trait.

Several methods have been developed to screen polymorphisms and some examples are listed below. The reference of Kwok and Chen (2003) and Kwok (2001) provide overviews of some of these methods; both of these references are specifically incorporated by reference.

SNPs or other polymorphisms relating to mtDNA position 10398 can be characterized by the use of any of these methods or suitable modification thereof. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or any other biochemical interpretation.

1. DNA Sequencing

The most commonly used method of characterizing a polymorphism is direct DNA sequencing of the genetic locus that flanks and includes the polymorphism. Such analysis can be accomplished using either the “dideoxy-mediated chain termination method,” also known as the “Sanger Method” (Sanger et al., 1975). Sequencing in combination with genomic sequence-specific amplification technologies, such as the polymerase chain reaction may be utilized to facilitate the recovery of the desired genes (Mullis et al., 1986; European Patent Application 50,424; European Patent Application. 84,796, European Patent Application 258,017, European Patent Application. 237,362; European Patent Application. 201,184; U.S. Pat. Nos. 4,683,202; 4,582,788; and 4,683,194), all of the above incorporated herein by reference.

2. Exonuclease Resistance

Other methods that can be employed to determine the identity of a nucleotide present at a polymorphic site utilize a specialized exonuclease-resistant nucleotide derivative (U.S. Pat. No. 4,656,127). A primer complementary to an allelic sequence immediately 3′-to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

3. Microsequencing Methods

Several other primer-guided nucleotide incorporation procedures for assaying polymorphic sites in DNA have been described (Komher et al., 1989; Sokolov, 1990; Syvanen 1990; Kuppuswamy et al., 1991; Prezant et al., 1992; Ugozzoll et al., 1992; Nyren et al., 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al., 1990).

4. Extension in Solution

French Patent 2,650,840 and PCT Application WO91/02087 discuss a solution-based method for determining the identity of the nucleotide of a polymorphic site. According to these methods, a primer complementary to allelic sequences immediately 3′-to a polymorphic site is used. The identity of the nucleotide of that site is determined using labeled dideoxynucleotide derivatives which are incorporated at the end of the primer if complementary to the nucleotide of the polymorphic site.

5. Genetic Bit Analysis or Solid-Phase Extension

PCT Application WO92/15712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

6. Oligonucleotide Ligation Assay (OLA)

This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR have also been described (Nickerson et al., 1990). Here PCR is used to achieve the exponential amplification of target DNA, which is then detected using the OLA.

7. Ligase/Polymerase-Mediated Genetic Bit Analysis

U.S. Pat. No. 5,952,174 describes a method that also involves two primers capable of hybridizing to abutting sequences of a target molecule. The hybridized product is formed on a solid support to which the target is immobilized. Here the hybridization occurs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

8. Invasive Cleavage Reactions

Invasive cleavage reactions can be used to evaluate cellular DNA for a particular polymorphism. A technology called INVADER® employs such reactions (e.g., de Arruda et al., 2002; Stevens et al., 2003, which are incorporated by reference). Generally, there are three nucleic acid molecules: 1) an oligonucleotide upstream of the target site (“upstream oligo”), 2) a probe oligonucleotide covering the target site (“probe”), and 3) a single-stranded DNA with the target site (“target”). The upstream oligo and probe do not overlap but they contain contiguous sequences. The probe contains a donor fluorophore, such as fluoroscein, and an acceptor dye, such as Dabcyl. The nucleotide at the 3′ terminal end of the upstream oligo overlaps (“invades”) the first base pair of a probe-target duplex. Then the probe is cleaved by a structure-specific 5′ nuclease causing separation of the fluorophore/quencher pair, which increases the amount of fluorescence that can be detected. See Lu et al. (2004). In some cases, the assay is conducted on a solid-surface or in an array format.

9. Other Methods to Detect SNPs

Several other specific methods for SNP detection and identification are presented below and may be used as such or with suitable modifications in conjunction with identifying polymorphisms (directly or indirectly) at mtDNA position 10398. Several other methods are also described on the SNP web site of the NCBI at the website on the World Wide Web at ncbi.nlm.nih.gov/SNP, incorporated herein by reference.

In a particular embodiment, extended sequence information may be determined at any given locus in a population, which allows one to identify exactly which SNPs will be redundant and which will be essential in association studies. In studies of genomic DNA material the latter is referred to as ‘haplotype tag SNPs (htSNPs),’ markers that capture the haplotypes of a gene or a region of linkage disequilibrium. See Johnson et al. (2001) and Ke and Cardon (2003), each of which is incorporated herein by reference, for exemplary methods.

The VDA-assay utilizes PCR amplification of genomic segments by long PCR methods using TaKaRa LA Taq reagents and other standard reaction conditions. The long amplification can amplify DNA sizes of about 2,000-12,000 bp. Hybridization of products to variant detector array (VDA) can be performed by a Affymetrix High Throughput Screening Center and analyzed with computerized software.

A method called Chip Assay uses PCR amplification of genomic segments by standard or long PCR protocols. Hybridization products are analyzed by VDA, Halushka et al. (1999), incorporated herein by reference. SNPs are generally classified as “Certain” or “Likely” based on computer analysis of hybridization patterns. By comparison to alternative detection methods such as nucleotide sequencing, “Certain” SNPs have been confirmed 100% of the time; and “Likely” SNPs have been confirmed 73% of the time by this method.

Other methods simply involve PCR amplification following digestion with the relevant restriction enzyme. Yet others involve sequencing of purified PCR products from known genomic regions.

In yet another method, individual exons or overlapping fragments of large exons are PCR-amplified. Primers are designed from published or database sequences and PCR-amplification of genomic DNA is performed using the following conditions: 200 ng DNA template, 0.5 μM each primer, 80 μM each of dCTP, dATP, dTTP and dGTP, 5% formamide, 1.5 mM MgCl₂, 0.5U of Taq polymerase and 0.1 volume of the Taq buffer. Thermal cycling is performed and resulting PCR-products are analyzed by PCR-single strand conformation polymorphism (PCR-SSCP) analysis, under a variety of conditions, e.g, 5 or 10% polyacrylamide gel with 15% urea, with or without 5% glycerol. Electrophoresis is performed overnight. PCR-products that show mobility shifts are reamplified and sequenced to identify nucleotide variation.

In a method called CGAP-GAI (DEMIGLACE), sequence and alignment data (from a PHRAP.ace file), quality scores for the sequence base calls (from PHRED quality files), distance information (from PHYLIP dnadist and neighbour programs) and base-calling data (from PHRED ‘-d’ switch) are loaded into memory. Sequences are aligned and examined for each vertical chunk (‘slice’) of the resulting assembly for disagreement. Any such slice is considered a candidate SNP (DEMIGLACE). A number of filters are used by DEMIGLACE to eliminate slices that are not likely to represent true polymorphisms. These include filters that: (i) exclude sequences in any given slice from SNP consideration where neighboring sequence quality scores drop 40% or more; (ii) exclude calls in which peak amplitude is below the fifteenth percentile of all base calls for that nucleotide type; (iii) disqualify regions of a sequence having a high number of disagreements with the consensus from participating in SNP calculations; (iv) removed from consideration any base call with an alternative call in which the peak takes up 25% or more of the area of the called peak; (v) exclude variations that occur in only one read direction. PHRED quality scores were converted into probability-of-error values for each nucleotide in the slice. Standard Baysian methods are used to calculate the posterior probability that there is evidence of nucleotide heterogeneity at a given location.

In a method called CU-RDF (RESEQ), PCR amplification is performed from DNA isolated from blood using specific primers for each SNP, and after typical cleanup protocols to remove unused primers and free nucleotides, direct sequencing using the same or nested primers.

In a method called DEBNICK (METHOD-B), a comparative analysis of clustered EST sequences is performed and confirmed by fluorescent-based DNA sequencing. In a related method, called DEBNICK (METHOD-C), comparative analysis of clustered EST sequences with phred quality>20 at the site of the mismatch, average phred quality>=20 over 5 bases 5′-FLANK and 3′ to the SNP, no mismatches in 5 bases 5′ and 3′ to the SNP, at least two occurrences of each allele is performed and confirmed by examining traces.

In a method identified by ERO (RESEW), new primers sets are designed for electronically published STSs and used to amplify DNA from 10 different mouse strains. The amplification product from each strain is then gel purified and sequenced using a standard dideoxy, cycle sequencing technique with ³³P-labeled terminators. All the ddATP terminated reactions are then loaded in adjacent lanes of a sequencing gel followed by all of the ddGTP reactions and so on. SNPs are identified by visually scanning the radiographs.

In another method identified as ERO (RESEQ-HT), new primers sets are designed for electronically published murine DNA sequences and used to amplify DNA from 10 different mouse strains. The amplification product from each strain is prepared for sequencing by treating with Exonuclease I and Shrimp Alkaline Phosphatase. Sequencing is performed using ABI Prism Big Dye Terminator Ready Reaction Kit (Perkin-Elmer) and sequence samples are run on the 3700 DNA Analyzer (96 Capillary Sequencer).

FGU-CBT (SCA2-SNP) identifies a method where the region containing the SNP were PCR amplified using the primers SCA2-FP3 and SCA2-RP3. Approximately 100 ng of genomic DNA is amplified in a 50 ml reaction volume containing a final concentration of 5 mM Tris, 25 mM KCl, 0.75 mM MgCl₂, 0.05% gelatin, 20 pmol of each primer and 0.5U of Taq DNA polymerase. Samples are denatured, annealed and extended and the PCR product is purified from a band cut out of the agarose gel using, for example, the QIAquick gel extraction kit (Qiagen) and is sequenced using dye terminator chemistry on an ABI Prism 377 automated DNA sequencer with the PCR primers.

In a method identified as JBLACK (SEQ/RESTRICT), two independent PCR reactions are performed with genomic DNA. Products from the first reaction are analyzed by sequencing, indicating a unique FspI restriction site. The mutation is confirmed in the product of the second PCR reaction by digesting with Fsp I.

In a method described as KWOK(1), SNPs are identified by comparing high quality genomic sequence data from four randomly chosen individuals by direct DNA sequencing of PCR products with dye-terminator chemistry (see Kwok et al., 2003). In a related method identified as KWOK(2) SNPs are identified by comparing high quality genomic sequence data from overlapping large-insert clones such as bacterial artificial chromosomes (BACs) or P1-based artificial chromosomes (PACs). An STS containing this SNP is then developed and the existence of the SNP in various populations is confirmed by pooled DNA sequencing (see Taillon-Miller et al., 1998). In another similar method called KWOK(3), SNPs are identified by comparing high quality genomic sequence data from overlapping large-insert clones BACs or PACs. The SNPs found by this approach represent DNA sequence variations between the two donor chromosomes but the allele frequencies in the general population have not yet been determined. In method KWOK(5), SNPs are identified by comparing high quality genomic sequence data from a homozygous DNA sample and one or more pooled DNA samples by direct DNA sequencing of PCR products with dye-terminator chemistry. The STSs used are developed from sequence data found in publicly available databases. Specifically, these STSs are amplified by PCR against a complete hydatidiform mole (CHM) that has been shown to be homozygous at all loci and a pool of DNA samples from 80 CEPH parents (see Kwok et al., 1994).

In another such method, KWOK (OverlapSnpDetectionWithPolyBayes), SNPs are discovered by automated computer analysis of overlapping regions of large-insert human genomic clone sequences. For data acquisition, clone sequences are obtained directly from large-scale sequencing centers. This is necessary because base quality sequences are not present/available through GenBank. Raw data processing involves analyzed of clone sequences and accompanying base quality information for consistency. Finished (‘base perfect’, error rate lower than 1 in 10,000 bp) sequences with no associated base quality sequences are assigned a uniform base quality value of 40 (1 in 10,000 by error rate). Draft sequences without base quality values are rejected. Processed sequences are entered into a local database. A version of each sequence with known human repeats masked is also stored. Repeat masking is performed with the program “MASKERAID.” Overlap detection: Putative overlaps are detected with the program “WUBLAST.” Several filtering steps followed in order to eliminate false overlap detection results, i.e., similarities between a pair of clone sequences that arise due to sequence duplication as opposed to true overlap. Total length of overlap, overall percent similarity, number of sequence differences between nucleotides with high base quality value “high-quality mismatches.” Results are also compared to results of restriction fragment mapping of genomic clones at Washington University Genome Sequencing Center, finisher's reports on overlaps, and results of the sequence contig building effort at the NCBI. SNP detection: Overlapping pairs of clone sequence are analyzed for candidate SNP sites with the ‘POLYBAYES’ SNP detection software.

Sequence differences between the pair of sequences are scored for the probability of representing true sequence variation as opposed to sequencing error. This process requires the presence of base quality values for both sequences. High-scoring candidates are extracted. The search is restricted to substitution-type single base pair variations. Confidence score of candidate SNP is computed by the POLYBAYES software.

In method identified by KWOK (TaqMan assay), the TaqMan assay is used to determine genotypes for numerous random individuals (e.g., 384). The techniques is designed to be used in the case of a diploid genome (i.e., nuclear genetic material) but may also be employed to analyze mtDNA sequences. In method identified by KYUGEN(Q1), DNA samples of indicated populations are pooled and analyzed by PLACE-SSCP. Peak heights of each allele in the pooled analysis are corrected by those in a heterozygote, and are subsequently used for calculation of allele frequencies. Allele frequencies higher than 10% are reliably quantified by this method. Allele frequency=0 (zero) means that the allele was found among individuals, but the corresponding peak is not seen in the examination of pool. Allele frequency=0-0.1 indicates that minor alleles are detected in the pool but the peaks are too low to reliably quantify.

In yet another method identified as KYUGEN, PCR products are post-labeled with fluorescent dyes and analyzed by an automated capillary electrophoresis system under SSCP conditions (PLACE-SSCP). Four or more individual DNAs are analyzed with or without two pooled DNA (Japanese pool and CEPH parents pool) in a series of experiments. Alleles are identified by visual inspection. Individual DNAs with different genotypes are sequenced and SNPs identified. Allele frequencies are estimated from peak heights in the pooled samples after correction of signal bias using peak heights in heterozygotes. For the PCR primers are tagged to have 5′-ATT or 5′-GTT at their ends for post-labeling of both strands. Samples of DNA (10 ng/μl) are amplified in reaction mixtures containing the buffer (10 mM Tris-HCl, pH 8.3 or 9.3, 50 mM KCl, 2.0 mM MgCl₂), 0.25 μM of each primer, 200 μM of each dNTP, and 0.025 units/μl of Taq DNA polymerase premixed with anti-Taq antibody. The two strands of PCR products are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. For the SSCP: an aliquot of fluorescently labeled PCR products and TAMRA-labeled internal markers are added to deionized formamide, and denatured. Electrophoresis is performed in a capillary using an ABI Prism 310 Genetic Analyzer. Genescan softwares (P-E Biosystems) are used for data collection and data processing. DNA of individuals (two to eleven) including those who showed different genotypes on SSCP are subjected for direct sequencing using big-dye terminator chemistry, on ABI Prism 310 sequencers. Multiple sequence trace files obtained from ABI Prism 310 are processed and aligned by Phred/Phrap and viewed using Consed viewer. SNPs are identified by PolyPhred software and visual inspection.

In yet another method identified as KYUGEN, individuals with different genotypes are searched by denaturing HPLC (DHPLC) or PLACE-SSCP (Inazuka et al., 1997) and their sequences are determined to identify SNPs. PCR is performed with primers tagged with 5′-ATT or 5′-GTT at their ends for post-labeling of both strands. DHPLC analysis is carried out using the WAVE DNA fragment analysis system (Transgenomic). PCR products are injected into DNASep column, and separated under the conditions determined using WAVEMaker program (Transgenomic). The two strands of PCR products that are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. SSCP followed by electrophoresis is performed in a capillary using an ABI Prism 310 Genetic Analyzer. Genescan softwares (P-E Biosystems). DNA of individuals including those who showed different genotypes on DHPLC or SSCP are subjected for direct sequencing using big-dye terminator chemistry, on ABI Prism 310 sequencer. Multiple sequence trace files obtained from ABI Prism 310 are processed and aligned by Phred/Phrap and viewed using Consed viewer. SNPs are identified by PolyPhred software and visual inspection. Trace chromatogram data of EST sequences in Unigene are processed with PHRED. To identify likely SNPs, single base mismatches are reported from multiple sequence alignments produced by the programs PHRAP, BRO and POA for each Unigene cluster. BRO corrected possible misreported EST orientations, while POA identified and analyzed non-linear alignment structures indicative of gene mixing/chimeras that might produce spurious SNPs. Bayesian inference is used to weigh evidence for true polymorphism versus sequencing error, misalignment or ambiguity, misclustering or chimeric EST sequences, assessing data such as raw chromatogram height, sharpness, overlap and spacing; sequencing error rates; context-sensitivity; cDNA library origin, etc.

In another method, overlapping human DNA sequences which contained putative insertion/deletion polymorphisms are identified through searches of public databases. PCR primers which flanked each polymorphic site are selected from the consensus sequences. Primers are used to amplify individual or pooled human genomic DNA. Resulting PCR products are resolved on a denaturing polyacrylamide gel and a PhosphorImager is used to estimate allele frequencies from DNA pools.

F. Mass Spectrometry

Another methods uses mass spectrometry to determine the time of flight of the different molecules containing different allelic variants (Sequenom MassArray). This approach, know as QGE, combines the advantages of microarrays and real-time PCR, allowing the expression levels of large numbers of genes to be accurately quantified. The new technology starts with competitive PCR, followed by mass spectroscopy to allow highly accurate measurement of an intrinsic physical property of a nucleic acid molecule: its mass. This approach can be used to study hundreds, and in some cases thousands, of genes in large numbers of samples

QGE method starts with competitive a PCR reaction that contains a defined amount of an internal control which calibrates the reaction. The QGE assay is designed such that the competitor oligonucleotide has an identical sequence to the gene-region of interest except for a single artificially introduced base change. The cDNA and the competitor are then amplified in the same reaction, thus subjecting them to the same conditions throughout the assay. Once the competitive PCR assay is completed, the cDNA and the competitor are assayed using a simple primer extension reaction in the presence of a mixture of ddNTPs and dNTPs. The extended primers are designed to have different masses so that the products from the cDNA and the competitor can be distinguished through mass spectroscopy. See the world-wide-web at sequenom.com.

G. Linkage Disequilibrium

Polymorphisms in linkage disequilibrium with the number of TA repeats may also be used with the methods of the present invention. “Linkage disequilibrium” (“LD” as used herein, though also referred to as “LED” in the art) refers to a situation where a particular combination of alleles (i.e., a variant form of a given gene) or polymorphisms at two loci appears more frequently than would be expected by chance. “Significant” as used in respect to linkage disequilibrium, as determined by one of skill in the art, is contemplated to be a statistical p or α value that may be 0.25 or 0.1 and may be 0.1, 0.05. 0.001, 0.00001 or less.

### V. MONITORING, PROPHYLAXIS AND PROGNOSIS

A. LQTS

1. Monitoring

Subjects with LQTS who have not developed symptoms of the disease are candidates for short and long term monitoring with implantable devices that record electrocardiographic activity (‘event recorders’). A demonstration of subclinical or occult cardiac rhythm disturbances or frank arrhythmias would contribute to the risk assessment.

2. Arrhythmia Prevention/Termination

Arrhythmia suppression involves the use of medications or surgical procedures that attack the underlying cause of the arrhythmias associated with LQTS. Since the cause of arrhythmias in LQTS is after depolarizations, and these after depolarizations are increased in states of adrenergic stimulation, steps can be taken to blunt adrenergic stimulation in these individuals.

Administration of β-receptor blocking agents decreases the risk of stress induced arrhythmias. β-blockers are the first choice in treating Long QT syndrome. In 2004 it has been shown that genotype and QT interval duration are independent predictors of recurrence of life-threatening events during β-blockers therapy. Specifically the presence of QTc>500ms and LQT2 and LQT3 genotype are associated with the highest incidence of recurrence. In these patients primary prevention with ICD (Implantable cardioverter-defibrillator) implantation can be considered.

Potassium supplementation is another preventative method. If the potassium content in the blood rises, the action potential shortens and due to this reason it is believed that increasing potassium concentration could minimize the occurrence of arrhythmias. It should work best in LQT2 since the HERG channel is especially sensible to potassium concentration, but the use is experimental and not evidence based.

Mexiletine is a sodium channel blocker. In LQT3, the problem is that the sodium channel does not close properly. Mexiletine closes these channels and is believed to be usable when other therapies fail. It should be especially effective in LQT3 but there is no evidence based documentation.

Amputation of the cervical sympathetic chain (left stellectomy) may be used as an add-on therapy to β-blockers but modern therapy mostly favors ICD implantation if beta blocker therapy fails.

Arrhythmia termination involves stopping a life-threatening arrhythmia once it has already occurred. The only effective form of arrhythmia termination in individuals with LQTS is placement of an implantable cardioverter-defibrillator (ICD). ICD are commonly used in patients with syncopes despite beta blocker therapy, and in patients who have experienced a cardiac arrest.

3. Prognosis

The risk for untreated LQTS patients having events (syncopes or cardiac arrest) can be predicted from their genotype (LQT1-8), gender and corrected QT interval.

High risk (>50%):
QTc>500 msec LQT1 & LQT2 & LQT3 (males)

Intermediate risk (30-50%):
QTc>500 msec LQT3 (females)
QTc<500 msec LQT2 (females)& LQT3

Low risk (<30%):
QTc<500 msec LQT1 & LQT2 (males)

B. SIDS

To reduce the likelihood of SIDS, parents of infants are encouraged to take several precautions in order to reduce the likelihood of SIDS.

Sleeping on the back has been recommended by (among others) the American Academy of Pediatrics (starting in 1992) to avoid SIDS. The incidence of SIDS has fallen sharply in a number of countries in which the back to bed recommendation has been widely adopted, such as the US and New Zealand. However, the absolute incidence of SIDS prior to the Back to Sleep Campaign was already dropping in the U.S., from 1.511 per 1000 in 1979 to 1.301 per 1000 in 1991.

Among the theories supporting the Back to Sleep recommendation is the idea that small infants with little or no control of their heads may, while face down, inhale their exhaled breath (high in carbon dioxide) or smother themselves on their bedding—the brain-stem anomaly research (above) suggests that babies with that particular genetic makeup do not react “normally” by moving away from the pooled CO₂, and thus smother. Another theory is that babies sleep more soundly when placed on their stomachs, and are unable to rouse themselves when they have an incidence of sleep apnea, which is thought to be common in infants.

Arguments against infant back-sleeping include concerns that an infant could choke on fluids it brings up. Hospital neonatal-intensive-care-unit (NICU) staff commonly place preterm newborns on their stomach, although they advise parents to place their infants on their backs after going home from the hospital. Other concerns raised about the Back to Sleep Campaign have included the possible increased risk of positional facial and head deformities (“positional plagiocephaly”), possible interference with development of good sleep habits (which in turn may have other adverse effects), and possible interference with motor skills development (as infants delay attempts to lift their heads, crawl, etc.).

A 2003 study, which investigated racial disparities in infant mortality in Chicago, found that previously or currently breastfeeding infants in the study had ⅕ the rate of SIDS compared with non-breastfed infants, but that “it became nonsignificant in the multivariate model that included the other environmental factors.” These results are consistent with most published reports and suggest that other factors associated with breastfeeding, rather than breastfeeding itself, are protective.” However, a more recent study shows that breast feeding reduces the risk of SIDS by approximately 50% at all infant ages.

Select studies suggest that limiting the amount of co-sleeping could lower a child's risk of SIDS. A 2005 policy statement by the American Academy of Pediatrics on sleep environment and the risk of SIDS deemed co-sleeping and bed sharing unsafe. One article reports that co-sleeping infants have a greater risk of airway covering than when the same infant sleeps alone in a cot.

Some data has suggested that almost all SIDS deaths in adult beds would be occurring when other prevention methods, such as placing infants on their backs, are not used. Co-sleeping studied in the West has been present mostly in poorer families where other risk factors are present. while co-sleeping in other cultures such as in China is more prevalent and is done in combination with practices such as sleeping children on their back, correlating with a significantly lower rate of SIDS than the West. There are also evolutionary theories as to why co-sleeping would be healthier for infants than sleeping alone. Further studies have suggested that factors associated with safe co-sleeping such as enhanced infant arousals are responsible for a positive contribution to SIDS prevention.

Depending on the child, co-sleeping may be made safer through the use of a bedside “co-sleeper.” Unattended adult beds are unsafe for infants, as are adult beds with excess bedding, intoxicated guardians, or those who smoke. Co-sleeping in couches is also very hazardous. Available evidence indicates that the safest place for infants to sleep is a crib in the parent's room.

According to the U.S. Surgeon General's Report, secondhand smoke is connected to SIDS. Infants who die from SIDS tend to have higher concentrations of nicotine and cotinine (a biological marker for secondhand smoke exposure) in their lungs than those who die from other causes. Infants exposed to secondhand smoke after birth are also at a greater risk of SIDS. Parents who smoke can significantly reduce their children's risk of SIDS by either quitting or smoking only outside and leaving their house completely smoke-free.

To prevent SIDS, product safety experts advise against using pillows, sleep positioners, bumper pads, stuffed animals, or fluffy bedding in the crib and recommend instead dressing the child warmly and keeping the crib “naked.” Infants' blankets should also not be placed over their heads. It has been recommended that infants should be covered only up to their chest with their arms exposed. This helps eliminate the chances of the infant shifting the blanket over his head.

In colder environments where bedding is required to maintain a baby's body temperature, the use of a “baby sleep bag” or “sleep sack” is becoming more popular. This is a soft bag with holes for the baby's arms and head. A zipper allows the bag to be closed around the baby. A study published in 1998 has shown the protective effects of a sleep sack as reducing the incidence of turning from back to front during sleep, reinforcing putting a baby to sleep on its back for placement into the sleep sack and preventing bedding from coming up over the face which leads to increased temperature and carbon dioxide rebreathing. They conclude that the use of a sleeping-sack should be particularly promoted for infants with a low birth weight. The American Academy of Pediatrics also recommends them as a type of bedding that warms the baby without covering its head.

According to a 2005 meta-analysis, most studies favor pacifier use. According to the American Academy of Pediatrics, pacifier use seems to reduce the risk of SIDS, although the mechanism by which this happens is unclear. SIDS experts and policy makers have not recommended the use of pacifiers to reduce the risk of SIDS because of several problems associated to pacifier use, like increased risk of otitis, gastrointestinal infections and oral colonization with Candida species. A recent study shows that pacifier use by breastfed infants does not reduce the rate of breastfeeding.

A 2005 study indicated that use of a pacifier is associated with up to a 90% reduction in the risk of SIDS depending on the ambiental factors, and it reduced the effect of other risk factors. It has been speculated that the raised surface of the pacifier holds the infant's face away from the mattress, reducing the risk of suffocation. If a postmortem investigation does not occur or is insufficient, a suffocated baby may be misdiagnosed with SIDS.

According to a study of nearly 500 babies published the October 2008 Archives of Pediatrics & Adolescent Medicine, using a fan to circulate air correlates with a lower risk of sudden infant death syndrome. Researchers took into account other risk factors and found that fan use was associated with a 72% lower risk of SIDS. Only 3% of the babies who died had a fan on in the room during their last sleep, the mothers reported. That compared to 12% of the babies who lived. Using a fan reduced risk most for babies in poor sleeping environments.

Bumper pads may be a contributing factor in SIDS deaths and should be removed. Health Canada, the Canadian government's health department, issued an advisory recommending against the use of bumper pads.

### VII. EXAMPLES

The following examples are included to demonstrate specific embodiments of the invention. It should be appreciated by those of skill in the art that the techniques disclosed in the examples that follow represent techniques discovered by the inventor to function well in the practice of the invention, and thus can be considered to constitute specific modes for its practice. However, those of skill in the art should, in light of the present disclosure, appreciate that many changes can be made in the specific embodiments that are disclosed and still obtain a like or similar result without departing from the spirit and scope of the invention.

### Example 1

### LQTS

### Materials and Methods

Study Population. The inventors studied an LQT1 South African founder population of mixed Dutch and French Huguenot origin harboring a mutation in KCNQ1 (A341V) (Brink et al., 2005). Cardiac events were defined as syncope (fainting spells with transient, but complete, loss of consciousness), aborted cardiac arrest (requiring resuscitation) and sudden cardiac death. Mutation carriers were classified as either symptomatic or asymptomatic. Symptomatic subjects were mutation carriers who experienced at least one cardiac event, whereas to be defined asymptomatic, a mutation carrier had to be at least 15 years old and not treated with β-adrenergic receptor antagonists. Additionally, symptomatic mutation carriers were classified by the severity of their clinical manifestations into two groups: those with a severe form of the disease (cardiac arrest and/or sudden cardiac death) and those with a milder form of the disease (symptomatic patients with no cardiac arrest/sudden cardiac death). Baseline electrocardiograms (ECGs) recorded in the absence of β-blocker therapy were coded and subsequently analyzed by one investigator (L.C.) blinded to genotype. Baseline heart rate (HR) and duration of the QT and RR intervals in leads II and V3 were measured from resting 12-lead ECGs. To allow QT values to be compared among subjects, the QT interval was corrected for heart rate (QTc) by using Bazett's formula.

All probands and family members provided written informed consent for clinical and genetic testing. Protocols were approved by the Ethical Review Boards of the Tygerberg Hospital of Stellenbosch University and the University of Pavia, and the Vanderbilt University Institutional Review Board. Approved consent forms were provided in English or Afrikaans as appropriate.

Genotyping. Genotyping of index cases and family members for the A341V mutation was previously described (Brink et al., 2005). The NOS1AP variants rs4657139, rs16847548, rs12567209, rs10494366, rs6683968 were genotyped using the 5′ nucleotidase TaqMan® assay (ABI Prism 7900HT, Applied Biosystems, Foster City, Calif.). Three of these variants (rs10494366, rs4657139, rs6683868) (Arking et al., 2006; Post et al., 2007; Newton-Cheh et al., 2007) were among the first to be tested for association with QT interval in general populations while the remaining two variants (rs16847548, rs12567209) were associated with sudden cardiac death in a community-based study (Kao et al., 2009). FIG. 1A provides the minor allele frequency (MAF) for each of the tested polymorphisms in this population and in the western European ancestry sample of the HapMap Project.

Statistical Analysis. Data are reported as mean and standard deviation (SD) for continuous variables; whenever the distribution was skewed, median, interquartile range (IQR) or quintiles were reported. Differences in baseline characteristics among groups of subjects were assessed with either a t-test or χ² test. Two-sided p-values<0.05 were considered statistically significant.

Association analyses were performed using the pedigree disequilibrium test (PDT) that allows the use of related trios and discordant sibpairs from extended pedigrees to identify associations of disease and marker (Martin et al., 2000). Extended pedigrees are ideal suited for analysis by PDT and the program is robust to any non-independence among pedigrees (Hardy et al., 2001). In the original description of this founder population, there were 22 extended pedigrees including up to 5 generations that could be genealogically linked (Brink et al., 2005). Because PDT can only handle up to 3-generation families, the inventors sub-divided the founder population into a series of 49 non-overlapping 3-generation pedigrees. Triads were then defined as informative nuclear families in which there is at least one affected child, both parents genotyped at the marker and at least one heterozygous parent. Discordant sibpairs were also informative if they had at least one affected and one unaffected sibling with different marker genotypes, with or without parental genotype data. Informative extended pedigrees contained at least one informative nuclear family and/or discordant sibship. Affectedness status of subjects was depended on the phenotype of interest: symptomatic mutation carriers, symptomatic mutation carriers with a severe form of the disease or mutation carriers with a prolonged QTc.

Because the inventors examined association with up to 5 variants, the inventors applied a correction for multiple testing based on the spectral decomposition (SpD) of matrices of the pairwise correlation coefficient (r) between variants (Nyholt, 2004; Li and Ji, 2005). This method estimates the effective number of independent markers (M_{eff-Li}) by taking account of the intermarker LD; the test criteria is then adjusted by the Bonferroni correction as though there were M_{eff-Li} independent tests. Using this approach, the inventors determined that there were 4 effectively independent tests among the 5 genotyped NOS1AP variants. Therefore, in the initial PDT association analysis between symptoms and NOS1AP genotype, the inventors used a corrected α-level of 0.0125 (0.05/4) as the threshold for statistical significance.

The inventors also carried out an empirical calculation of the type I error level, which is not dependent on any explicit or hidden statistical assumptions of the PDT method. Using the extensive computer simulation facility developed by one of the authors (Pedpower, D. A. G.), a computer simulation randomly assigned marker genotypes to the exact family structures of the families in the data set. Marker and disease loci were simulated to be biallelic, and the loci were in linkage equilibrium. The relationship of the markers to the disease locus represented the null hypothesis, that is, there was no association between the disease and the marker. The inventors simulated 10,000 such data sets and showed that the false positive rate followed a χ² distribution. Thus, the particular characteristics of this data set represented no unusual or confounding problems to the PDT.

Statistical calculations were performed by using STATA 10 (StataCorp, College Station, Tex. 77845 USA) and the PDT software (world-wide-web at chg.duke.edu/research/pdt.html). Linkage disequilibrium (LD) across NOS1AP was evaluated using Haploview (world-wide-web at broad.mit.edu/mpg/haploview) using the HapMap data for this region (world-wide-web at hapmap.org). The r² correlation coefficient (FIG. 1B) and the normalized disequilibrium coefficient (D′) were used as a measure of LD (Gabriel et al., 2002).

### Results

Study Population. The inventors studied a South African LQT1 (KCNQ1-A341V mutation) population that consisted of 500 family members of whom 205 were mutation carriers, 228 were non-carriers, and 67 were not genetically tested. For this study, DNA samples were available on 255 subjects. There was no sex bias (females 47%; males 53%). Among the 205 mutation carriers, there were 174 subjects that had a clearly defined phenotype status. Thirty mutation carriers were classified as asymptomatic and were older than 15 years and not treated with β-adrenergic receptor antagonists (β-blockers), while 9 other subjects without symptoms were too young (age<15) to be classified (Brink et al., 2005). Among the 165 subjects with a defined and classifiable phenotype, 135 had symptoms (82%) (syncope with transient but complete loss of consciousness, aborted cardiac arrest requiring resuscitation or sudden cardiac death) with a median age at first cardiac event of 6 years (IQR 4-10). Among the 135 symptomatic subjects, 56 suffered cardiac arrest and/or sudden cardiac death, and the remaining 79 symptomatic mutation carriers had only syncope. These findings are consistent with the unusual severity of this particular mutation as demonstrated by the inventors' prior analysis of 21 unrelated families from 8 different countries all carrying the KCNQ1-A341V mutation (Crotti et al., 2007). One hundred-nine mutation carriers and 101 non-carriers with a resting ECG recorded in the absence of β-blocker therapy were analyzed for differences in QTc interval. Baseline QTc was longer in mutation carriers than in non-carriers (487±44 vs 402±23 ms, p<0.001) with no significant differences in mean age at the time of ECG recording or distribution of males and females between the two groups. Despite sharing the same genetic defect, mutation-carriers exhibited a wide range of QTc (397-676 ms).

Association between NOS1AP and clinical manifestations. NOS1AP variants were genotyped in 255 individuals (143 mutation carriers including 135 with a classifiable phenotype and 8 subjects younger than 15 years, and 112 non-carriers) grouped into 49 three-generation pedigrees derived from the founder population. The inventors analyzed the association between symptoms and NOS1AP genotype using the pedigree-disequilibrium test (PDT) in 30 informative pedigrees including 29 informative triads and 102 informative discordant sibpairs that were selected by the PDT software. Two NOS1AP variants exhibited differential transmission when evaluated for association with the occurrence of cardiac symptoms (rs4657139, PDT p=0.019; rs16847548, PDT p=0.003; Table 2). After correction for multiple hypothesis testing (see Methods, above), only the minor allele of rs16847548 (C allele) remained significantly associated with an increased risk of cardiac events. However, these two variants (rs4657139, rs16847548) were only 6 kb apart and are in LD (D′=1, r²=0.36) among Caucasian subjects of western European ancestry genotyped by the HapMap project. Three other NOS1AP variants (rs12567206, rs10494366, rs6683968) were not significantly associated with symptoms in the South African LQTS population. The non-associated variants were either distant from the other two NOS1AP variants (rs10494366, rs6683968) or exhibited very low minor allele frequency (rs12567206; FIGS. 1A-B). The two markers with high r² had similar MAF (31% and 32%; FIGS. 1A-B). The non-associated variants were either distant from the other two markers in NOS1AP (rs10494366, rs6683968) or exhibited very low minor allele frequency (rs12567206; FIGS. 1A-B). The two markers with high r² had MAF values of 31% and 32% (FIGS. 1A-B).

TABLE 2
Pedigree Dysequilibrium Test for the Association Between NOS1AP
Variants and Symptoms in South African LQTS Population
Association with any symptoms

| | | Allele Count | | | | | |
|---|---|---|---|---|---|---|---|
| | | Triads (parental | | | | | |
| | | contribution) | | | | | |
| | | | Not | Discordant sib pairs | | | |
| | | Transmitted | transmitted | Affected | Unaffected | PDT | |
| SNP | Allele | (n = 58*) | (n = 58) | (n = 56 | (n = 64) | Statistic | p-value |
| rs4657139 | A | 26 | 16 | 39 | 33 | 5.500 | 0.019 |
| | T | 32 | 42 | 73 | 95 | | |
| rs16847548 | C | 17 | 5 | 26 | 18 | 48.643 | 0.03 |
| | T | 41 | 53 | 86 | 110 | | |

Association with severe symptoms

| | | Allele Count | | | | | |
|---|---|---|---|---|---|---|---|
| | | Triads (parental | | | | | |
| | | contribution) | | | | | |
| | | | Not | Discordant sib pairs | | | |
| | | Transmitted | transmitted | Affected | Unaffected | PDT | |
| SNP | Allele | (n = 26) | (n = 26) | (n = 9) | (n = 10) | Statistic | p-value |
| rs4657139 | A | 15 | 7 | 11 | 7 | 4.829 | 0.28 |
| | T | 11 | 19 | 7 | 13 | | |
| rs16847548 | C | 8 | 1 | 9 | 5 | 6.000 | 0.014 |
| | T | 18 | 25 | 9 | 15 | | |

*number of subjects given in parentheses

The inventors further tested whether the NOS1AP risk alleles at rs4657139 and rs16847548 were associated with the occurrence of severe cardiac events (cardiac arrest, sudden death) among symptomatic KCNQ1-A341V mutations carriers. In this analysis, rs4657139 and rs16847548 were both significantly associated with the risk of life-threatening events (rs4657139, PDT p=0.028; rs16847548, PDT p=0.014) suggesting that NOS1AP variants modify risk for life-threatening cardiac events in this Afrikaner LQTS population. The inventors could not compute a relative risk for life-threatening events caused by the presence of the risk allele because mutation carriers are related, which produces a risk that is biased upwards. However, an odds ratio (OR) can be considered the upper bound of the risk calculated using unrelated symptomatic subjects. With that caveat, mutation carriers with at least one copy of the minor allele at rs16847548 or rs4657139 have a 1.4 (95% C.I. 0.76-2.6) or 1.8 times (95% C.I. 1.1-3.3) greater chance of having life-threatening events than the mutation carriers without the minor allele, respectively.

Association between NOS1AP and QT interval. The inventors also tested for association between the two NOS1AP variants that were associated with symptoms and the QTc. They examined allele sharing between two groups of KCNQ1-A341V mutation carriers defined by the upper and lower 40% of QTc values. They did not consider the central quintile in this analysis to avoid the inclusion of a confounding “grey area.” Therefore, this analysis only included mutation carriers with QTc≦472 ms or QTc>492 ms as measured by a resting electrocardiogram in the absence of β-blockers (n=118) to avoid the confounding effects of treatment. Among 21 informative pedigrees included in this analysis, there were 14 informative triads and 49 informative discordant sibpairs.

Minor alleles of the two NOS1AP variants associated with symptoms were significantly associated with a QTc greater than 492 ms in the population, (rs4657139, PDT p=0.03; rs16847548, PDT p=0.03; Table 3) which is consistent with the effect of these variants on QTc observed in healthy populations (Arking et al., 2006; Aarnoudse et al., 2007; Post et al., 2007; Raitakari et al., 2008; Tobin et al., 2008; Lehtinen et al., 2008; Arking et al., 2009; Eijgelsheim et al., 2009; Newton-Cheh et al., 2009; Pfeufer et al., 2009). Importantly, QTc prolongation associated with NOS1AP was observed in subjects despite an already markedly prolonged QTc interval.

TABLE 3
Pedigree Dysequilibrium Test for the Association Between NOS1AP
Variants and QTc Interval (QTc ≧ 493 ms vs QTc ≦ 472 ms) in KCNQ1-A341V
Mutation Carriers

| | | Allele Count | | | | | |
|---|---|---|---|---|---|---|---|
| | | Triads (parental | | | | | |
| | | contribution) | | | | | |
| | | | Not | Discordant sib pairs | | | |
| | | Transmitted | transmitted | QTc ≧ 493 | QTc ≧ 493 | PDT | |
| SNP | Allele | (n = 28*) | (n = 28) | (n = 25) | (n = 37) | Statistic | p-value |
| rs4657139 | A | 13 | 6 | 19 | 22 | 4.626 | 0.03 |
| | T | 15 | 22 | 31 | 52 | | |
| rs16847548 | C | 7 | 1 | 12 | 15 | 4.754 | 0.03 |
| | T | 21 | 27 | 38 | 59 | | |

*number of subjects given in parentheses

In conclusion, the inventors demonstrated a significant association between common variants in NOS1AP and the clinical severity of LQTS with special reference to life-threatening arrhythmias. The association of NOS1AP genetic variants with risk for life-threatening arrhythmias points to NOS1AP as a genetic modifier of LQTS and this knowledge should become clinically useful for risk-stratification after validation in other LQTS populations.

### Example 2

### SIDS

### Materials and Methods

Given the inventors' finding that common variants in NOS1AP increase clinical severity of LQTS, they next tested the possibility that they may also modify the risk of SIDS. One hundred twenty-seven Norwegian SIDS cases that did not carry LQTS gene mutations and 180 ethnically-matched controls were genotyped for two single nucleotide polymorphisms (rs10494366, rs16847548) located in intron 1 of NOS1AP. Genotyping was performed using the 5′ nucleotidase TaqMan assay method.

### Results

The NOS1AP variant rs10494366 was significantly associated with the risk of SIDS (p=0.015), while no association was observed for rs16847548. Specifically, subjects carrying the GG or TG genotype (G is the minor allele for rs10494366) had a 1.8 greater risk of SIDS compared to subject carrying the TT genotype (OR=1.8; 95% CI 1.1-2.8; p=0.015).

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference.
U.S. Pat. No. 5,840,873
U.S. Pat. No. 5,843,640
U.S. Pat. No. 5,843,650
U.S. Pat. No. 5,843,651
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,487
U.S. Pat. No. 5,849,497
U.S. Pat. No. 5,849,546
U.S. Pat. No. 5,849,547
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,905,024
U.S. Pat. No. 5,910,407
U.S. Pat. No. 5,912,124
U.S. Pat. No. 5,912,145
U.S. Pat. No. 5,916,776
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,582,788
U.S. Pat. No. 4,656,127
U.S. Pat. No. 4,683,194
U.S. Pat. No. 4,683,195
U.S. Pat. No. 4,683,202
U.S. Pat. No. 4,800,159
U.S. Pat. No. 4,938,948
U.S. Pat. No. 4,946,773
U.S. Pat. No. 5,021,236
U.S. Pat. No. 5,196,066
U.S. Pat. No. 5,279,721
U.S. Pat. No. 5,843,663
U.S. Pat. No. 5,849,481
U.S. Pat. No. 5,849,483
U.S. Pat. No. 5,849,486
U.S. Pat. No. 5,851,770
U.S. Pat. No. 5,851,772
U.S. Pat. No. 5,866,337
U.S. Pat. No. 5,900,481
U.S. Pat. No. 5,912,148
U.S. Pat. No. 5,916,779
U.S. Pat. No. 5,919,626
U.S. Pat. No. 5,925,525
U.S. Pat. No. 5,928,870
Aarnoudse et al., Circulation, 116:10-16, 2007.
Abbondanzo et al., Breast Cancer Res. Treat., 16:182(151), 1990.
Allred et al., Arch. Surg., 125(1):107-113, 1990.
Arking et al., Nature Genet., 38:644-651, 2006.
Arking et al., PLoS ONE, 4:e4333, 2009.
Atherton et al., Biol. Reprod., 32(1):155-171, 1985.
Ausubel et al., In: Current Protocols in Molecular Biology, John, Wiley & Sons, Inc, NY, 1989.
Becker et al., Br. J. Clin. Pharmacol., 67:61-67, 2009.
Bellus, J., Macromol. Sci. Pure Appl. Chem., A31(1): 1355-1376, 1994.
Berberian et al., Science, 261:1588-1591, 1993.
Brink et al., Circulation, 112:2602-2610, 2005.
Brown et al. Immunol. Ser., 53:69-82, 1990.
Campbell, In: Monoclonal Antibody Technology, Laboratory Techniques in Biochemistry and Molecular Biology, Burden and Von Knippenberg (Eds.), Elseview, Amsterdam, 13:71-74/75-83, 1984.
Chang et al., Proc. Natl. Acad. Sci. USA, 105:4477-4482, 2008.
Chugh et al., Circulation, 119:663-670, 2009.
Cleary et al., J. Biol. Chem., 269(29):18747-9, 1994.
Crotti et al., Circulation, 112:1251-1258, 2005.
Crotti et al., Circulation, 116:2366-2375, 2007.
de Arruda et al., Expert. Rev. Mol. Diagn., 2:487-496, 2002.
De Jager et al., Semin. Nucl. Med., 23(2):165-179, 1993.
Dholakia et al., J. Biol. Chem., 264(34):20638-20642, 1989.
Doolittle and Ben-Zeev, Methods Mol Biol., 109:215-237, 1999.
Eijgelsheim et al., Hum. Mol. Genet., 18:347-357, 2009.
European Patent 201,184
European Patent 237,362
European Patent 258,017
European Patent 320 308
European Patent 329 822
European Patent Appln. 50,424
European Patent Appln. 84,796
French Patent 2,650,840
Frohman, In: PCR Protocols: A Guide To Methods And Applications, Academic Press, N.Y., 1990.
Gabriel et al., Science, 296:2225-2229, 2002.
Gefter, et al., Somatic Cell Genet., 3:231-236, 1977.
Goding, In: Monoclonal Antibodies: Principles and Practice, 2d ed., Academic Press, FL., 60-66, and 71-74, 1986.
Great Britain Appln. 2 202 328
Gulbis and Galand, Hum. Pathol., 24(12):1271-1285, 1993.
Halushka et al., Nat. Genet., 22(3):239-247, 1999.
Hardy et al., Genet. Epidemiol., 21(Suppl 1):S441-S446, 2001.
Harlow and Lane, In: Antibodies: A Laboratory Manual, Cold Spring Harbor Laboratory, Cold Spring Harbor, N.Y., 346-348, 1988.
Inazuka et al., Genome Res, 7(11):1094-1103, 1997.
Innis et al., Proc. Natl. Acad. Sci. USA, 85(24):9436-9440, 1988.
Johnson et al., Nat. Genet., 29(2):233-237, 2001.
Kang et al., Science, 240:1034-1036, 1988.
Kao et al., Circulation, 119:940-951, 2009.
Ke and Cardon, Bioinformatics, 19(2):287-288, 2003.
Khatoon et al., Ann. of Neurology, 26, 210-219, 1989.
King et al., J. Biol. Chem., 269, 10210-10218, 1989.
Kohler and Milstein, Eur. J. Immunol., 6:511-519, 1976.
Kohler and Milstein, Nature, 256:495-497, 1975.
Kohler et al., Methods Enzymol., 178:3, 1989.
Komher et al., Nucl. Acids. Res. 17:7779-7784, 1989.
Kreier et al., In: Infection, Resistance and Immunity, Harper & Row, New York, 1991.
Kuppuswamy et al., Proc. Natl. Acad. Sci. USA, 88:1143-1147, 1991.
Kwoh et al., Proc. Natl. Acad. Sci. USA, 86:1173, 1989.
Kwok and Chen, Curr Issues Mol. Biol., April; 5(2):43-60, 2003.
Kwok et al., Genomics, 23(1):138-144, 1994.
Kwok et al., Methods Mol. Biol., 212:71-84, 2003.
Kwok, Annu. Rev. Genomics Hum. Genet., 2:235-258, 2001.
Landegren, et al., Science 241:1077-1080, 1988.
Lehtinen et al., Diabetes, 57:1108-1114, 2008.
Lenert et al., Science, 248(4963):1639-1643, 1990.
Li and Ji, Heredity, 95:221-227, 2005.
Lu et al., Biopolymers, 73:606-613, 2004.
Martin et al., Am. J. Hum. Genet., 67:146-154, 2000.
Maxam et al., Proc. Natl. Acad. Sci. USA, 74:560, 1977.
Moss et al., Circulation, 115:2481-2489, 2007.
Mullis et al., Cold Spring Harbor Symp. Quant. Biol. 51:263-273, 1986.
Nakamura et al., In: Handbook of Experimental Immunology (4^{th} Ed.), Weir et al., (eds). 1:27, Blackwell Scientific Publ., Oxford, 1987.
Newton-Cheh et al., BMC Med. Genet., 8(Suppl I):S7, 2007.
Newton-Cheh et al., Nature Genet., 41:399-406, 2009.
Nickerson et al., Proc. Natl. Acad. Sci. USA, 87:8923-8927, 1990.
Nyholt, Am. J. Hum. Genet., 74:765-769, 2004.
Nyren et al., Anal. Biochem. 208:171-175, 1993.
Ohara et al., Proc. Natl. Acad. Sci. USA, 86:5673-5677, 1989.
O'Shannessy et al., J. Immun. Meth., 99, 153-161, 1987.
Owens & Haley, J. Biol. Chem., 259:14843-14848, 1987.
PCT Appln. PCT/US87/00880
PCT Appln. PCT/US89/01025
PCT Appln. WO 88/10315
PCT Appln. WO 89/06700
PCT Appln. WO 91/02087
PCT Appln. WO 92/15712
Pfeufer et al., Nature Genet., 41:407-414, 2009.
Post et al., Hum. Hexed., 64:214-219, 2007.
Potter & Haley, Meth. in Enzymol., 91, 613-633, 1983.
Prezant et al., Hum. Mutat., 1:159-164, 1992.
Priori et al., Circulation, 99:529-533, 1999.
Priori et al., N Engl. J. Med., 348:1866-1874, 2003.
Raitakari et al., Ann. Med., 41:144-151, 2008.
Sambrook and Russell, Molecular Cloning: A Laboratory Manual 3^{rd} Ed., Cold Spring Harbor Laboratory Press, 2001.
Sanger et al., J. Molec. Biol., 94:441, 1975.
Sasso et al., J. Immunol., 142:2778-2783, 1989.
Schwartz and Crotti, In: Long QT and short QT syndrome, Zipes and Jalife (Eds.), Philadelphia, Elsevier/Saunders, 5^{th} Ed., 731-744, 2009.
Schwartz and Wolf, Circulation. 57:1074-1077, 1978.
Schwartz et al., Circulation, 103:89-95, 2001
Schwartz et al., J. Cardiovasc. Electrophysiol., 14:1120-1121, 2003.
Shorki et al., J. Immunol., 146:936-940, 1991.
Silvermann et al., J. Clin. Invest., 96:417-426, 1995.
Sokolov, Nucl. Acids Res. 18:3671, 1990.
Stevens et al., Biotechniques, 34:198-203, 2003.
Syvanen et al., Genomics 8:684-692, 1990.
Taillon-Miller et al., Genome Res, 8(7):748-754, 1998.
Tobin et al., Int. J. Epidemiol., 37:1132-1141, 2008.
Ugozzoll et al., GATA, 9:107-112, 1992.
Walker et al., Proc. Natl. Acad. Sci. USA, 89:392-396 1992.
Wang et al., Nature Genet., 12:17-23, 1996.
Westenskow et al., Circulation, 109:1834-1841, 2004.
Wratten et al., Am. J. Psychiatry, 166:434-441, 2009.

What is claimed is:

1. A method of predicting increased risk of sudden death from long QT syndrome (LQTS) or sudden infant death syndrome (SIDS) comprising:
(a) providing a DNA-containing sample from a subject with congenital LQTS;
(b) assessing the structure of the NOS1AP gene at rs16847548 and/or rs4657139; and
(c) making a prediction of risk based on the structure of the NOS1AP gene at rs16847548 and/or rs4657139;
wherein the presence of a rs16847548 C allele and/or a rs4657139 A allele indicates that said subject is at increased risk of experiencing sudden death from LQTS or SIDS as compared to a subject having a rs16847548 T allele and/or a rs4657139 T allele.

2. The method of claim 1, further comprising examining at least one additional risk factor for LQTS in for the subject.

3. The method of claim 2, wherein the at least one additional risk factor for LQTS is presence of a mutation in an LQTS risk gene or a LQTS score of 3 or more.

4. The method of claim 2, wherein the at least one additional risk factor for LQTS is a relative diagnosed with LQTS.

5. The method of claim 1, wherein assessing the structure comprises sequencing.

6. The method of claim 1, wherein assessing the structure comprises primer extension.

7. The method of claim 1, wherein assessing the structure comprises differential hybridization.

8. The method of claim 1, wherein assessing the structure comprises a 5′-nucleotidase assay.

9. The method of claim 1, further comprising amplifying at least a portion of the NOS1AP gene.

10. The method of claim 9, wherein amplifying comprises polymerase chain reaction.

11. The method of claim 1, further comprising making a decision regarding monitoring of said subject.

12. The method of claim 11, wherein monitoring comprises implantation of an automated internal defibrillator or internal recording device (‘event recorder’).

13. The method of claim 1, wherein the subject is a newborn of less than about one month of age.

14. The method of claim 1, wherein the subject is an infant of about one month to about 3 years of age.

15. The method of claim 1, wherein the subject is an adult.

16. The method of claim 1, wherein the subject has a rs16847548 C allele and a rs4657139 A allele.

17. The method of claim 1, wherein the subject has a rs16847548 T allele and a rs4657139 T allele.

18. The method claim 1, wherein the subject has a rs16847548 C allele and a rs4657139 T allele.

19. The method of claim 1, wherein the subject has a rs16847548 T allele and a rs4657139 A allele.

20. The method of claim 1, further comprising treating the subject when determined to be at increased risk of sudden death with an anti-arrhythmic compound.

21. The method of claim 20, wherein the anti-arrythmic compound is a β blocker, a sodium channel blocker, or potassium channel modulator.

22. The method of claim 1, further comprising diagnosing said the subject as having LQTS.

23. The method of claim 22, wherein diagnosing comprises genetic testing for an LQTS mutation in DNA from the subject.

24. The method of claim 22, wherein diagnosing comprises taking a family history from the subject.

25. The method of claim 22, wherein diagnosing comprises a physical examination of the subject.

26. The method of claim 25, wherein the physical examination comprises an electrocardiogram.

27. The method of claim 1, wherein assessing comprises assessing a structure that is determined to be in linkage disequilibrium with rs16847548 and/or rs4657139.
